(19) United States
(12) Patent Application Publication
(10) Pub. No.: US 2013/0023520 A1
(43) Pub. Date: Jan. 24, 2013

(54) ANAESTHETIC EYE SOLUTION AND METHOD OF USE

(71) Applicant: Amir Sahba Jalali, Columbia, MO (US)

(72) Inventor: Amir Sahba Jalali, Columbia, MO (US)

(21) Appl. No.: 13/189,526

(22) Filed: Jul. 24, 2011

(51) Int. Cl. A61K 31/4436; A61K 31/245; A61K 31/167; A61K 31/196; A61P 7/00; A61P 31/00; A61P 29/00; A61P 27/14; A61P 9/00; A61P 35/00; A61K 31/55; A61P 27/02

(52) U.S. Cl. 514/214.02; 514/324; 514/217.05; 514/537; 514/626; 514/570

(57) ABSTRACT

An eye drop solution includes an anaesthetic and at least one medicament. The anaesthetic is preferably provided in a range of from about 0.001% to about 1.0%. The eye drop solution may further include either a lubricant or a preservative, or both of these.

### RELATED APPLICATIONS

N/A

### BACKGROUND OF THE INVENTION

1. Field of the Invention

The present invention relates generally to a composition for addressing irritation of the eye, including irritation of the corneal surface. More specifically, the present invention relates to an eye drop solution including an anaesthetic component, and to a method of treating or preventing eye irritation using the same.

2. Background of the Invention

The cornea is the clear front surface of the eye. It includes the epithelium, stroma, and endothelium. The epithelium is the outer-most layer and is made up of cells that have the ability to multiply and regenerate injured portions of the epithelium. The epithelium also includes nerve-endings that are able to feel the presence of dust and other irritants on the surface of the eye.

The surface of the eye may become irritated in a variety of ways. One common source of ocular surface maladies is dryness. A number of eye drop solutions are available for addressing dryness. Other sources of irritation include various chemical compounds, medicaments, and the like that are introduced onto the surface of the eye for therapeutic purposes. The surface of the eye may also be irritated physically, such as, for example, by the use of hard contact lenses.

Despite the availability of soft contact lenses, hard lenses remain in common use. Gas permeable hard lenses are generally considered healthier than hydrogel contact lenses, and for some refractive errors they provide clearer, more precise visual acuity. In some cases, soft lenses may not be able to provide the visual correction a patient needs. Hard lenses are also more resistant to deposits and bacteria than soft lenses. Along with the advantages of hard lenses, however, comes the disadvantage that hard lenses can irritate a user's eye to a greater degree than soft lenses, particular upon initial insertion of the hard lens into the eye, during the adaptation period.

Chemical solutions may be applied to the surface of the eye for a number of reasons. Such solutions may be used for lubrication, or to address allergic reactions that have affected the eye. Prescription eye drop solutions may be used to introduce medicaments onto the surface of the eye. Such prescription eye drops represent a significant market within the eye care industry. Over an initial five-year period starting at product launch, the prescription eye drop solution Restasis® brought in over $1.5 billion in revenue. Many such eye drop solutions, whether over the counter or prescription, irritate the eye, particularly upon the initial application of the solution to the eye. The burning or other irritation experienced upon initial application of the solution to the eye can lead to a lack of prescription compliance among patients, and to a general lack of use of eye drop solutions by the public at large.

### SUMMARY OF THE INVENTION

The present invention provides an eye drop solution including an anaesthetic and at least one medicament. The anaesthetic component is preferably provided in a range of from about 0.001% to about 1.0% of the solution.

In another aspect of the invention, the eye drop solution includes antibiotics, non-steroidal anti-inflammatory drugs, antihistamines, vasoconstrictors, angiostatic steroids, anti-inflammatory steroids, anti-neovascularization steroids, or combinations of these.

In another aspect of the invention, the eye drop solution includes a lubricant, which may be, but is not limited to, glycerine, propylene glycol, mineral oil, hydroxypropyl methycellulose, hypromellose, carboxylmethylcellulose, povidone, polyvinyl alcohol, or combinations of these.

In another aspect of the invention, the eye drop solution includes a preservative, which may be, but is not limited to, benzalkonium chloride.

In another aspect of the invention, the anaesthetic may be lidocaine, proparacaine, tetracaine, or combinations of these.

In still another aspect of the invention, an eye drop solution includes from about 0.5% to about 1.0% lidocaine, a medicament, and a lubricant.

In still another aspect of the invention, an eye drop solution includes from about 0.1% to about 0.2% proparacaine, a medicament, and a lubricant.

In another aspect of the invention, an eye drop solution further includes from about 0.01% to about 0.02% ketotifen, from about 0.02% to about 0.04% epinastine HCL, from about 0.05% to about 0.08% bromfenac, from about 0.025% to about 0.04% azelastine, or combinations of these.

### DETAILED DESCRIPTION OF THE PREFERRED EMBODIMENTS

As used herein, a therapeutically effective amount refers to an amount of a compound, medicament, or other composition necessary to provide a desired benefit. The precise amount may vary depending on the particular composition of the solution at issue, and may vary among patients. In general, when referring to the anaesthetic component of the present invention, a therapeutically effective amount is preferably from about 0.001% to about 1.0% anaesthetic in solution. This range may be applied to any anaesthetic disclosed herein, as well as to any other suitable ophthalmic anaesthetic, whether or not specifically disclosed herein.

The present invention provides anaesthetic eye drop solutions for preventing or treating eye discomfort caused by a variety of factors. It is contemplated that the principles of the present invention may be applied to modify existing eye solutions, thereby introducing an anaesthetic component into the solutions. In addition, the present invention provides a number of eye drop solutions set forth herein that include an anaesthetic component. It is further contemplated that an eye drop solution of the present invention that includes an anaesthetic solution may be used prior to or in conjunction with existing eye drop solutions in order to treat or prevent irritation associated with the use of those existing solutions. The present solutions may also be used to treat or prevent pain or discomfort associated with physical contact on the eye, such as from hard contact lenses.

The eye drop solutions of the present invention preferably includes from about 0.001% to about 1.0% of a topical anaesthetic, in addition to other components, when desired, that impart other properties to the eye drop solutions. An exemplary anaesthetic used in eye care is proparacaine hydrochloride, which is generally available as a 0.5% solution. Proparacaine hydrochloride acts by stabilizing the neuronal membrane by binding to and inhibiting voltage-gated sodium channels. This inhibits sodium ion influx that is needed for the initiation and conduction of impulses within the neuronal cell. The end result is a loss of sensation. Proparacaine hydrochloride is currently used to numb the ocular surface for various procedures performed by healthcare practicioners. The anaesthetic has not been used in eye drop solutions provided over the counter or for patient use on a prescription basis. It is generally considered in the art to be too dangerous to allow patients to use such eye drops due to the dangers of over-anaesthetization. The formulations of the present invention provide anaesthetic and other effects to the eye while unexpectedly avoiding the dangers of over-anaesthetization.

An exemplary eye drop solution of the present invention may be provided, for example, by mixing a 0.5% proparacaine hydrochloride solution with sterile distilled water to achieve a final solution having from about 0.001% to about 0.4% proparacaine hydrochloride. Although proparacaine hydrochloride is a preferred anaesthetic compound, it is contemplated that any anaesthetic compound suitable for use on the ocular surface may be used. Other anaesthetics that may be used in conjunction with the present invention include tetracaine and lidocaine. Further, it is contemplated that a combination of anaesthetics may be used in any given eye drop solution of the present invention.

In addition to providing an eye drop solution that includes only proparacaine hydrochloride in a water base, it is contemplated that a variety of other agents known in the art may be provided along with the anaesthetic component of the present solutions.

The present eye drop solutions may include one or more lubricant, such as glycerin, propylene glycol, mineral oil, petroleum products, hydroxypropyl methylcellulose, hypromellose, carboxyl methylcellulose sodium, povidone, polyvinyl alcohol, or combinations of these.

The eye drop solutions of the present invention may include isotonicities such as D-mannitol, glucose, glycerin, or combinations of these.

Further, the present eye drop solutions may include buffering agents such as sodium dihydrogen phosphate, sodium hydrogen phosphate, potassium dihydrogen phosphate, potassium hydrogen phosphate, boric acid, sodium borate, citric acid, tartaric acid, sodium tartrate, and such combinations of these as are suitable.

The eye drop solutions of the present invention may also include pH adjusters to produce a final solution having a pH suitable for use as a topical eye solution. Such pH adjusters may include hydrochloric acid, acetic acid, sodium hydroxide, potassium hydroxide, and the like. Conventional acids and bases are well-known pH adjusters within the art.

For some solutions of the present inventions, medicaments that are poorly soluble in water may be included along with the anaesthetic component of the present eye drop solutions. In such cases, solubilizers may also be provided as needed, and may be chosen for properties providing solubility of the specific medicament in question. Solubilizers that may be used include polyoxyethylene glycol ethers, polyethylene glycol higher fatty acid esters, and polyoxyethylene fatty acid esters. Examples of components that may require the addition of solubilizers to the present formulations include non-steroidal anti-inflammatory agents (NSAIDS) such as, for example, naproxin; diclofenac; celecoxib (Celebrex®, Pfizer); sulindac; diflunisal; piroxicam; indomethacin; etodolac; meloxicam; ibuprofen; ketoprofen; r-flurbiprofen (Myriad Genetics, Inc.); mefenamic; nabumetone; tolmetin, and sodium salts of each of the foregoing; ketorolac bromethamine; ketorolac tromethamine (Acular®, Allergan, Inc.); choline magnesium trisalicylate; rofecoxib; valdecoxib; lumiracoxib; etoricoxib; aspirin; salicylic acid and its sodium salt; salicylate esters of α,β,γ-tocopherols and tocotrienols (and all their d, l, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; and phenylbutazone.

Eye solutions of the present invention may also include an antihistamine component. Suitable antihistamines include olopatadine and its hydrochloride, sulfate, or phosphate salt forms; fexofenadine and its hydrochloride, sulfate, or phosphate salt; azelastine and its hydrochloride, sulfate, or phosphate forms; diphenhydramine and its hydrochloride, sulfate, or phosphate forms; and promethazine and its hydrochloride, sulfate, or phosphate forms.

Some embodiments of the present invention may include angiostatic and/or anti-inflammatory steroids such as anecortive acetate (Retaane®, Alcon, Inc., Fort Worth, Tex.); tetrahydrocortisol; 4,9(11)-pregnadien-17α,21-diol-3,20-dione (Anecortave) and its -21-acetate salt; 11-epicortisol; 17α-hydroxyprogesterone; tetrahydrocortexolone; cortisona; cortisone acetate; hydrocortisone; hydrocortisone acetate; fludrocortisone; fludrocortisone acetate; fludrocortisone phosphate; prednisone; prednisolone; prednisolone sodium phosphate; methylprednisolone; methylprednisolone acetate; methylprednisolone, sodium succinate; triamcinolone; triamcinolone-16,21-diacetate; triamcinolone acetonide and its -21-acetate, -21-disodium phosphate, and -21-hemisuccinate forms; triamcinolone benetonide; triamcinolone hexacetonide; fluocinolone and fluocinolone acetate; dexamethasone and its 21-acetate, -21-(3,3-dimethylbutyrate), -21-phosphate disodium salt, -21-diethylaminoacetate, -21-isonicotinate, -21-dipropionate, and -21-palmitate forms; betamethasone and its -21-acetate, -21-adamantoate, -17-benzoate, -17,21-dipropionate, -17-valerate, and -21-phosphate disodium salts; beclomethasone; beclomethasone dipropionate; diflorasone; diflorasone diacetate; mometasone furoate; and acetazolamide (Diamox®, Lederle Parenterals, Inc., Carolina, Puerto Rico; several other manufacturers).

In addition, anti-neovascularization steroids may be provided as a components of the present invention. Such steroids include 21-nor-5β-pregnan-3α,17α,20-triol-3-acetate; 21-nor-5α-pregnan-3α,17α,20-triol-3-phosphate; 21-nor-5β-pregn-17(20)en-3α,16-diol; 21-nor-5β-pregnan-3α,17β,20-triol; 20-acetamide-21-nor-5α-pregnan-3α,17α-diol-3-acetate; 313 acetamido-5β-pregnan-11β,17α,21-triol-20-one-21-a-cetate; 21-nor-5α-pregnan-3α,17β,20-triol; 21α-methyl-5β-pregnan-3α,11β,17α,21-tetrol-20-1-one-21-methyl ether; 20-azido-21-nor-5β-pregnan-3α,17α-diol; 20(carbethoxymethyl)thio-21-nor-5β-pregnan-3α,17α-diol; 20-(4-fluorophenyl)thio-21-nor-5β-pregnan-3α,17α-diol; 16α-(2-hydroxyethyl)-17β-methyl-5β-androstan-3α,17α-diol; 20-cyano-21-nor-5β-pregnan-3α,17α-diol; 17α-methyl-51-androstan-3α,17β-diol; 21-nor-5β-pregn-17(20)en-3α-ol; 21- or -5β-pregn-17(20)en-3α-ol-3-acetate; 21-nor-5-pregn-17(20)-en-3α-ol-16-acetic acid 3-acetate; 3β-azido-5β-pregnan-11β,17α,21-triol-20-one-21-acetate; and 5β-pregnan-11β,17α,21-triol-20-one; 4-androsten-3-one-17β-carboxylic acid; 17α-ethynyl-5(10)-estren-17β-ol-3-one; and 17α-ethynyl-1,3,5(10)-estratrien-3,17β-diol.

The eye drop solutions of the present invention may also include hyperosmotic solutions that are pH balanced for the natural tears of the eye. Hyperosmotics may include mannitol, glycerin, isosorbide, or combinations of these. Further, vasoconstrictors such as naphazoline or brimonidine tartrate (for example, 0.05%) may be used in conjunction with the present eye drop formulations.

It is contemplated that with respect to each of the optional components presented above, which may be used in conjunction with the anaesthetic solution of the present invention, the listed components are exemplary and are not meant to be limiting. Addition components of each type listed are known in the art and may also be used in conjunction with the present invention. Further, it is contemplated that as new components of each type become known, those new components may also be used in conjunction with the present anaesthetic solution.

The present invention also provides for methods of using the anaesthetic eye drop solutions provided herein to reduce or eliminate pain or discomfort associated with other chemical components of an eye drop solution. It is preferred that the present anaesthetic solution is combined with compounds providing other desired effects to produce a single solution for use by a patient. For example, for a patient in need of an antihistamine eye drop solution, it is preferred that the present anaesthetic eye drop solution is provided with an antihistamine, or mixed with an antihistamine solution, to provide a single solution having from about 0.001% to about 1.0% anaesthetic (such as proparacaine hydrochloride), as well as a therapeutically-effective amount of an antihistamine solution. The amount of any given antihistamine solution needed in order to provide a therapeutically effectively amount is known in the art.

### Example 1

Table 1, below, provides an exemplary ophthalmic solution prepared in accordance with the teachings of the present invention. In the example provided, lidocaine provides the anaesthetic functionality of the solution. Ketotifen is an antihistamine and is used in the exemplary ophthalmic preparation to prevent eye irritation and itchiness commonly associated with seasonal allergies. Glycerine and propylene glycol serve as lubricants. Benzalkonium chloride (BAK) is a preservative.

TABLE 1

| Compound | Percentage (v) |
|---|---|
| Lidocaine | 0.5% |
| Ketotifen | 0.01% |
| Glycerine | 0.4% |
| Propylene glycol | 0.5% |
| Benzalkonium chloride | (effective percentage ranges are |
| | generally known in the art) |

### Example 2

Table 2, below, provides an alternative formulation for an ophthalmic solution having the properties of the solution provided in Example 1.

TABLE 2

| Compound | Percentage (v) |
|---|---|
| Lidocaine | 1.0% |
| Ketotifen | 0.02% |
| Glycerine | 0.6% |
| Polyethylene glycol | 0.2% |
| BAK | (effective percentage ranges are |
| | generally known in the art) |

### Example 3

Table 3, below, provides another exemplary ophthalmic solution prepared in accordance with the teachings of the present invention. In the example provided, proparacaine provides the anaesthetic functionality of the solution. Epinastine HCL is an antihistamine and carboxymethylcellulose serves as a lubricant. Bromfenac is a non-steroidal anti-inflammatory drug (NSAID).

TABLE 3

| Compound | Percentage (v) |
|---|---|
| Proparacaine | 0.1% |
| Epinastine HCL | 0.02% |
| Carboxymethylcellulose | 0.25% |
| Bromfenac | 0.05% |

### Example 4

Table 4, below, provides a variant formulation having a similar functionality to the solution provided in table 3, above.

TABLE 4

| Compound | Percentage (v) |
|---|---|
| Proparacaine | 0.2% |
| Epinastine HCL | 0.04% |
| Carboxymethylcellulose | 0.4% |
| Bromfenac | 0.08% |

### Example 5

Table 5, below, provides another exemplary ophthalmic solution prepared in accordance with the teachings of the present invention. In the example provided, proparacaine provides the anaesthetic functionality of the solution. Azelastine is a histamine antagonist. Glycerine and carboxymethylcellulose serve as lubricants.

TABLE 5

| Compound | Percentage (v) |
|---|---|
| Proparacaine | 0.15% |
| Azelastine | 0.025% |
| Carboxymethylcellulose | 0.25% |
| Glycerine | 0.4% |

### Example 6

Table 6, below, provides a variant formulation having a similar functionality to the solution provided in table 3, above.

TABLE 6

| Compound | Percentage (v) |
|---|---|
| Proparacaine | 0.25% |
| Azelastine | 0.04% |
| Carboxymethylcellulose | 0.4% |
| Glycerine | 0.6% |

Whether the present invention is utilized in the formulation of eye drop solutions including, in addition to the anaesthetic component, lubricants, steroid solutions, various medicaments, or other components, it is preferred that a single solution is produced for the ease of use of the patient. The anaesthetic quality of the present invention inhibits or prevents the irritation, burning, or other sensation often experienced with the use of eye drop solutions having these components, thereby reducing or eliminating discomfort to the patient and increasing patient compliance. It is contemplated, however, that in some instances two or more separate solutions may be used. This may occur, for example, when a patient does not have access to a combined solution having all of the chemical properties necessary to, or desired by, the patient. In such an instance, the patient may utilize a solution of the present invention having an anaesthetic component (and, if necessary or desired, other components as well) immediately prior to use of another eye drop solution. The use of the present solution will temporarily desensitize the eye and the patient may use the second solution with lessened discomfort or with no discomfort at all.

In addition to a method of using the present invention to reduce or eliminate discomfort associated with the chemical components of various eye drop solutions, the present invention also provides a method of using the present invention to reduce or eliminate discomfort or pain associated with physical effects on the eye, such as through the use of hard contact lenses. In such situations, a patient using hard contact lenses may, in conjunction with inserting the hard lenses onto the eye, introduce a solution of the present invention into the eye. The solution of the present invention will include an anaethestic component as described above, as well as, optionally, other components needed or desired by the patient. The temporary numbing of the cornea by use of a solution of the present invention will lessen or eliminate the discomfort associated with introducing the hard contact lens onto the eye.

Having thus described the preferred embodiment of the invention, what is claimed as new and desired to be protected by Letters Patent includes the following:

1. An eye drop solution comprising:
an anaesthetic; and
at least one medicament.

2. The eye drop solution of claim 1 wherein said anaesthetic is present in an amount as from about 0.001% to about 1.0% of the eye drop solution.

3. The eye drop solution of claim 1 wherein said at least one medicament is selected from the group consisting of antibiotics, non-steroidal anti-inflammatory drugs, antihistamines, vasoconstrictors, angiostatic steroids, anti-inflammatory steroids, anti-neovascularization steroids, and combinations thereof.

4. The eye drop solution of claim 1 further comprising a lubricant.

5. The eye drop solution of claim 4 wherein the lubricant is selected from the group consisting of glycerine, propylene glycol, mineral oil, hydroxypropyl methycellulose, hypromellose, carboxylmethylcellulose, povidone, polyvinyl alcohol, and combinations thereof.

6. The eye drop solution of claim 1 wherein said anaesthetic is selected from the group consisting of proparacaine, tetracaine, lidocaine, and combinations thereof.

7. An eye drop solution comprising:
from about 0.5% to about 1.0% lidocaine;
at least one medicament; and
a lubricant.

8. The eye drop solution of claim 7, further comprising a preservative.

9. The eye drop solution of claim 8, wherein the preservative is benzalkonium chloride.

10. The eye drop solution of claim 7 wherein the at least one medicament is selected from the group consisting of from about 0.01% to about 0.02% ketotifen, from about 0.02% to about 0.04% epinastine HCL, from about 0.05% to about 0.08% bromfenac, from about 0.025% to about 0.04% azelastine, and combinations thereof.

11. An eye drop solution comprising:
from about 0.01% to about 0.25% proparacaine;
at least one medicament; and
a lubricant.

12. The eye drop solution of claim 11 wherein the at least one medicament is selected from the group consisting of from about 0.01% to about 0.02% ketotifen, from about 0.02% to about 0.04% epinastine HCL, from about 0.05% to about 0.08% bromfenac, from about 0.025% to about 0.04% azelastine, and combinations thereof.
